(19) United States
(12) Patent Application Publication
(10) Pub. No.: US 2009/0240186 A1
(43) Pub. Date: Sep. 24, 2009

(54) Wound rebuilding band-aid without need of stitches

(76) Inventor: Hsu-Cheng Fang, Taichung County (TW)

Correspondence Address: Hsu-Cheng Fang, P.O.Box 44-2049, Taipei, 10668, TW

(21) Appl. No.: 12/232,775

(22) Filed: Sep. 24, 2008

(30) Foreign Application Priority Data
Mar. 19, 2008 (TW) 097204660

(51) Int. Cl. A61F 13/02; A61B 17/03

(52) U.S. Cl. 602/54; 606/216

(57) ABSTRACT

A band-aid includes at least one piece which includes a first adhesive portion and a second adhesive portion defined in two ends of an inner surface thereof. A lifting portion is located between the first and second adhesive portions. The first and second adhesive portions are adhered to two edges of a wound and the pulled toward each other by lifting the lifting portion. The two edges of the wound are rebuilt to reduce any possible scar by using the band-aid.

### BACKGROUND OF THE INVENTION

(1) Field of the Invention

The present invention relates to a band-aid and more particularly, to a band-aid to rebuild two edges of the wound without need of stitches.

The present invention intends to provide a band-aid which includes two adhesive portions on two ends thereof so as to pull the two edges of the wound toward each other to minimize the gap between the two edges such that the scar can be avoided.

(2) Description of the Prior Art

A conventional band-aid is used for small wound and includes a pad including proper medicine so as to prevent infection to the wound. When the wound is made by cut and a long wound is formed, the doctor stitch the two edges of the wound to keep the two edges contact such that there will be less visible scars formed after the wound is healed. However, to stitch the wound require certain conditions such as professional medical personals, professional skills such as the use of narcotic, etc., which may not available when dealing with the wound.

### SUMMARY OF THE INVENTION

The present invention relates to a band-aid which comprises at least one piece having an outer surface and an inner surface, a first adhesive portion and a second adhesive portion are defined in two ends of the inner surface. An lifting portion is located between the first and second adhesive portions. The first and second adhesive portions are adhered to two edges of a wound and the pulled toward each other by lifting the lifting portion, such that the two edges are quickly rebuilt to reduce the risk of forming a scar.

The primary object of the present invention is to provide a band-aid that includes two adhesive portions and an lifting portion is connected between the two adhesive portions. The two edges are pulled toward each other by lifting the lifting portion so that the edges are kept to contact each other during healing process.

The present invention will become more obvious from the following description when taken in connection with the accompanying drawings which show, for purposes of illustration only, a preferred embodiment in accordance with the present invention.

### BRIEF DESCRIPTION OF THE DRAWINGS

FIG. 1 is shows the first embodiment of the band-aid of the present invention;

FIG. 2 shows that the first embodiment of the band-aid of the present invention is applied to the wound;

FIG. 3 shows that the lifting portion of the first embodiment of the band-aid of the present invention is lifted;

FIG. 4 shows that the lifting portion of the first embodiment of the band-aid of the present invention is cut;

FIG. 5 is a perspective view to show the second embodiment of the band-aid of the present invention;

FIG. 6 is a plane view to show the second embodiment of the band-aid of the present invention;

FIG. 7 shows that the second embodiment of the band-aid of the present invention is applied to the wound;

FIG. 8 shows that the lifting portion of the second embodiment of the band-aid of the present invention is lifted;

FIG. 9 shows that the lifting portion of the second embodiment of the band-aid of the present invention is bent and adhered to the third adhesive portion;

FIG. 10 shows that the lifting portion of the second embodiment of the band-aid of the present invention is cut;

FIG. 11 is a plane view to show the third embodiment of the band-aid of the present invention;

FIG. 12 is a plane view to show the fourth embodiment of the band-aid of the present invention;

FIG. 13 is a plane view to show the fifth embodiment of the band-aid of the present invention;

FIG. 14 shows that the fifth embodiment of the band-aid of the present invention is applied to a wound;

FIG. 15 is a plane view to show the sixth embodiment of the band-aid of the present invention, and FIG. 16 shows that the sixth embodiment of the band-aid of the present invention is applied to a wound.

### DETAILED DESCRIPTION OF THE PREFERRED EMBODIMENTS

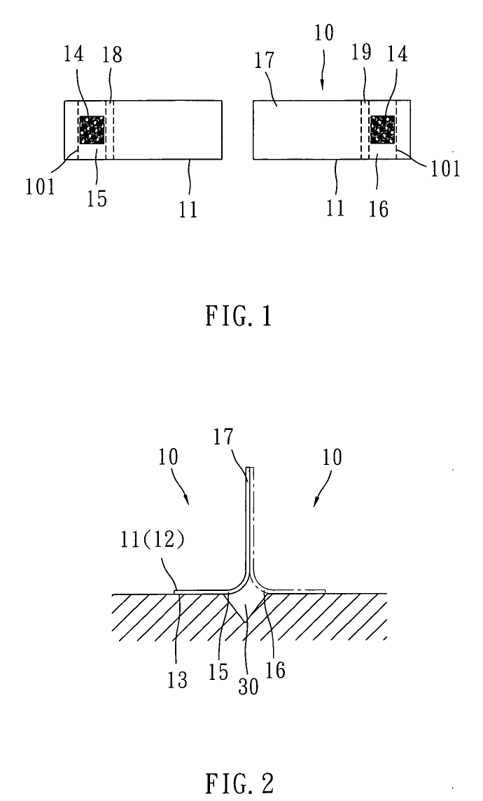

Referring to FIGS. 1 to 4, the first embodiment of the band-aid 10 of the present invention comprises two pieces 11 and each piece 11 includes an outer surface 12 and an inner surface 13. A first adhesive portion 15 and a second adhesive portion 16 are defined in two respective ends of the two pieces 11 respectively and the rest portion of each of the two pieces 11 is the lifting portion 17. When arranging the two pieces 11 as shown in FIG. 1, the lifting portion 17 is located between the first and second adhesive portions 15, 16. Two pads 14, 14 are located on the first and second adhesive portions 15, 16. A non-adhesive surface 18 is defined beside the pad 14 on the first adhesive portion 15 and located close to the lifting portion 17. Another non-adhesive surface 19 is defined beside the pad 14 on the second adhesive portion 16 and located close to the lifting portion 17. Two outer lines 101 are located beside the two pads 14 on the first and second adhesive portions 15, 16 respectively, the outer lines 101 are located remote from the lifting portion 17.

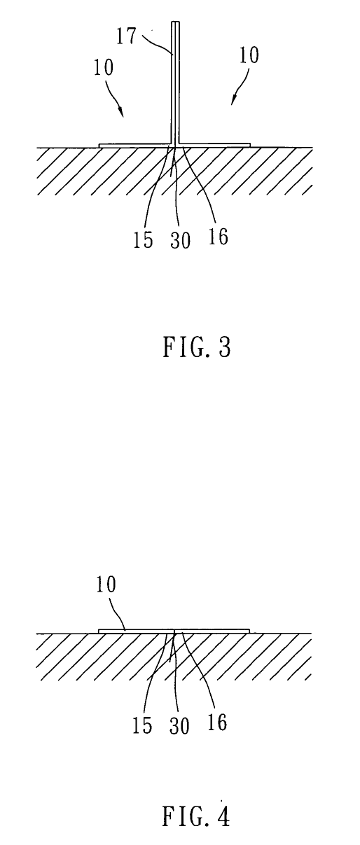

When applying the band-aid to the wound 30, the first and second adhesive portions 15, 16 are adhered to two edges of the wound and the lifting portions 17 are then pulled upward to move the two edges of the wound toward each other as shown in FIG. 3. The two edges are in contact with each other by lifting the lifting portions 17 and the lifting portions 17 are then cut as shown in FIG. 4. The contact edges are easily rebuilt to reduce the formation of scar. When removing the band-aid 10, the user peels the ends having the outer lines 101 and removes the two pieces 11 from the wound 30. It is noted that the band-aid 10 is applied to the wound transverse to the wound such that when removing the band-aid 10, the wound will not be torn apart again.

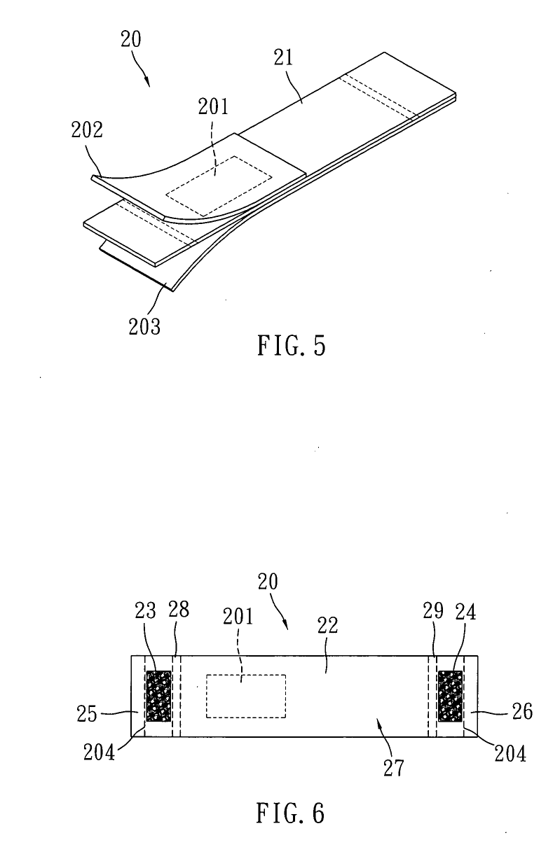

FIGS. 5 and 6 show a second embodiment of the band-aid 20 which is a single piece and a first adhesive portion 25 and a second adhesive portion 26 are located on two ends of an inner surface 22 thereof. A lifting portion 27 is located between the first and second adhesive portions 25, 26. Two pads 23, 24 are located on the first and second adhesive portions 25, 26. A non-adhesive surface 28 is defined beside the pad 23 on the first adhesive portion 25 and located close to the lifting portion 27. Another non-adhesive surface 29 is defined beside the pad 24 on the second adhesive portion 26 and located close to the lifting portion 27. Two outer lines 204 are located beside the two pads 23, 24 on the first and second adhesive portions 25, 26 respectively. The outer lines 204 are located remote from the lifting portion 27. A third adhesive portion 201 is defined in an outer surface 21 of the band-aid 20 and located corresponding to the lifting portion 27.

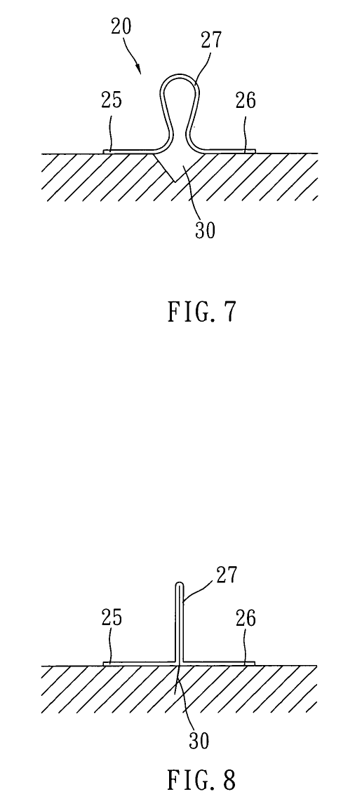

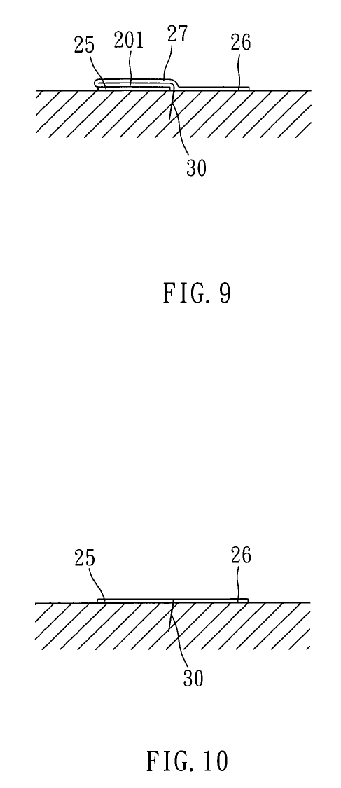

As shown in FIGS. 7 to 9, when applying the band-aid 20 to the wound 30, the first and second adhesive portions 25, 26 are adhered to two edges of the wound and the lifting portion 27 is pulled upward by hand or a tool to move the two edges of the wound toward each other as shown in FIG. 8. The two edges are in contact with each other by lifting the lifting portion 27 and the lifting portion 27 is bent and adhered to the third adhesive portion 201. It is noted that two releasing sheets 202, 203 are located on the outer surface 21 and the inner surface 22, and are removed when using the band-aid 20. Of course, the lifting portion 27 can be cut as shown in FIG. 10.

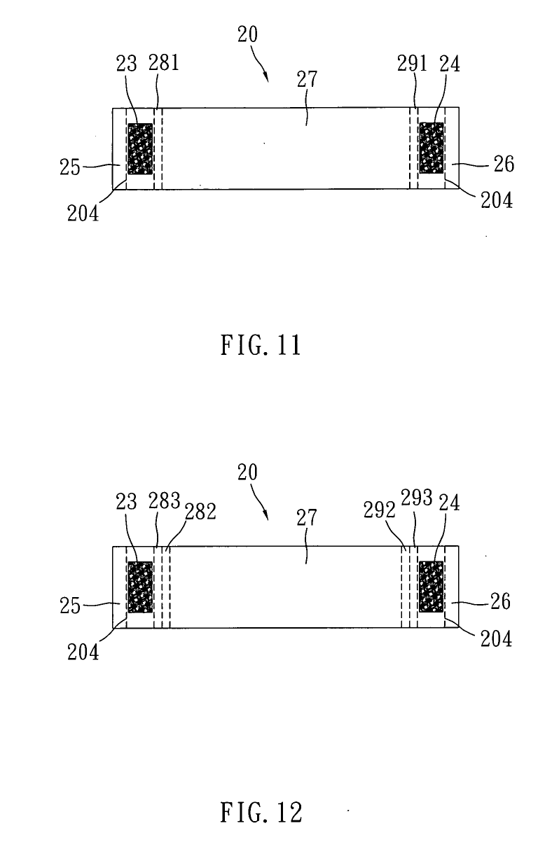

FIG. 11 shows a third embodiment of the band-aid 20 wherein an adhesive surface 281 is defined beside the pad 23 on the first adhesive portion 25 and located close to the lifting portion 27, and another adhesive surface 291 is defined beside the pad 24 on the second adhesive portion 26 and located close to the lifting portion 27. The two adhesive surfaces 281, 291 are adhered to each other when pulling the two edges of the wound together to ensure that the two edges are moved close to each other and/or in contact with each other.

FIG. 12 shows a fourth embodiment of the band-aid 20 wherein an adhesive surface 282 and a non-adhesive surface 283 are located side by side and defined beside the pad 23 on the first adhesive portion 25, the non-adhesive surface 283 is located right beside the pad 23 and the adhesive surface 282 and the non-adhesive surface 283 are close to the lifting portion 27. Another adhesive surface 292 and another non-adhesive surface 293 are located side by side and defined beside the pad 24 on the second adhesive portion 26, the non-adhesive surface 293 is located right beside the pad 24 and the adhesive surface 292 and the non-adhesive surface 293 are close to the lifting portion 27.

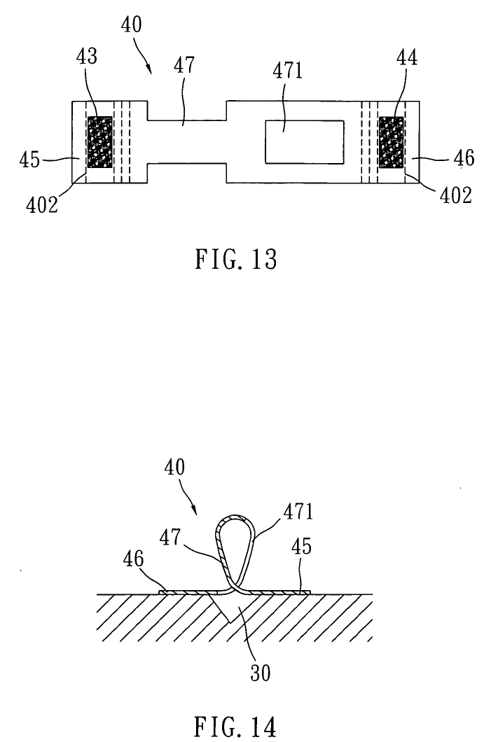

FIGS. 13 and 14 show the fifth embodiment of the band-aid 40 which is similar to the fourth embodiment and includes the an adhesive surface and a non-adhesive surface side by side and located beside the two respective pads 43, 44. Two outer lines 402 are defined in the two ends of the single piece of the band-aid 40. The main difference is that the lifting portion 47 of the band-aid 40 includes a narrowed neck portion and an opening 471 is defined through the lifting portion 47. When using the band-aid 40, the end with the first adhesive portion 45 and the pad 43 is extended through the opening 471 to adhere the two edges of the wound 30 by the two adhesive portions 45, 46. The lifting portion 47 is then lifted to move the two edges toward each other.

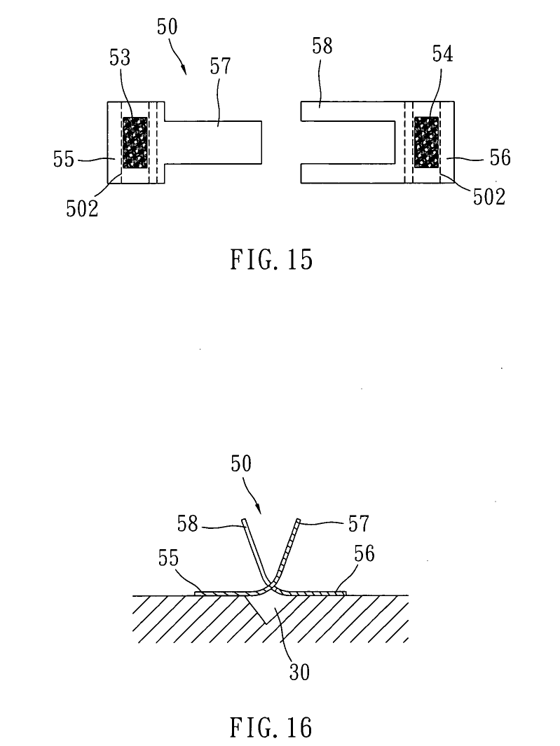

FIGS. 15 and 16 show the sixth embodiment of the band-aid 50 of the present invention, wherein the band-aid 50 includes two pieces and the lifting portion 57 of one of the two pieces is an elongate strip and the lifting portion 58 of the other piece includes a recess which is sized that the elongate strip is engaged with the recess. Both of the elongate strip and the U-shaped lifting portion 58 includes adhesive surfaces. The piece having the elongate strip has the first adhesive portion 55 with a pad 53 and an outer line 502 is defined beside the pad 53. The piece having the recess has the second adhesive portion 56 with a pad 54 and another outer line 502 is defined beside the pad 54. When using the band-aid 50, the elongate strip is pulled and adhered to the outer surface of the piece having the second adhesive portion 56 and extends through the recess of the other piece. The lifting portion 58 with the recess is pulled and adhered to the outer surface of the piece having the first adhesive portion 55. The two edges of the wound are then pulled toward each other and the two pieces are smoothly on the skin.

While we have shown and described the embodiment in accordance with the present invention, it should be clear to those skilled in the art that further embodiments may be made without departing from the scope of the present invention.

What is claimed is:

1. A band-aid comprising:
at least one piece having an outer surface and an inner surface which has a first adhesive portion and a second adhesive portion on two ends thereof, a lifting portion located between the first and second adhesive portions, the first and second adhesive portions adapted to be adhered to two edges of a wound and the being pulled toward each other by lifting the lifting portion.

2. The band-aid as claimed in claim 1, wherein the band-aid includes two pieces and the two first and second adhesive portions are located on the two pieces respectively.

3. The band-aid as claimed in claim 1, wherein the band-aid is a single piece.

4. The band-aid as claimed in claim 1, wherein a third adhesive portion is defined in the outer surface of the at least one piece and located corresponding to the lifting portion, the lifted lifting portion is bent and adhered to the third adhesive portion.

5. The band-aid as claimed in claim 1, wherein two pads are located on the first and second adhesive portions.

6. The band-aid as claimed in claim 5, wherein a non-adhesive surface is defined beside the pad on the first adhesive portion and located close to the lifting portion, another non-adhesive surface is defined beside the pad on the second adhesive portion and located close to the lifting portion.

7. The band-aid as claimed in claim 5, wherein an adhesive surface is defined beside the pad on the first adhesive portion and located close to the lifting portion, another adhesive surface is defined beside the pad on the second adhesive portion and located close to the lifting portion.

8. The band-aid as claimed in claim 5, wherein an adhesive surface and a non-adhesive surface are located side by side and defined beside the pad on the first adhesive portion, the non-adhesive surface is located right beside the pad and the adhesive surface and the non-adhesive surface are close to the lifting portion, another adhesive surface and another non-adhesive surface are located side by side and defined beside the pad on the second adhesive portion, the non-adhesive surface is located right beside the pad and the adhesive surface and the non-adhesive surface are close to the lifting portion.

9. The band-aid as claimed in claim 1, wherein two outer lines are located beside the first and second adhesive portions respectively, the outer lines are located remote from the lifting portion.

10. The band-aid as claimed in claim 1, wherein the lifting portion of the at least one piece includes a narrowed neck portion and an opening is defined through the lifting portion.

11. The band-aid as claimed in claim 2, wherein the lifting portion of one of the two pieces is an elongate strip and the lifting portion of the other piece is a U-shaped lifting portion and includes a recess which is sized that the elongate strip is engaged with the recess, both of the elongate strip and the U-shaped lifting portion includes adhesive surfaces.
